(19) United States
(12) Patent Application Publication
(10) Pub. No.: US 2004/0191187 A1
(43) Pub. Date: Sep. 30, 2004

(54) Multivalent ion compatible carbomer formulations

(76) Inventors: Hong Luo, Akron, OH (US); William Robert Wilber, Twinsburg, OH (US); Jian-Hwa Guo, Hudson, OH (US)

Correspondence Address: NOVEON IP HOLDINGS CORP., 9911 BRECKSVILLE ROAD, CLEVELAND, OH, 44141-3247, US

(21) Appl. No.: 10/766,274

(22) Filed: Jan. 28, 2004

Related U.S. Application Data
(60) Provisional application No. 60/444,042, filed on Jan. 31, 2003.

(51) Int. Cl. A61K 7/16

(52) U.S. Cl. 424/49

(57) ABSTRACT

A polymeric formulation, and more specifically, a toothpaste formulation, is disclosed containing a highly carboxylated polymer or copolymer derived from one or more unsaturated carboxylic acids in combination with a calcium carbonate abrasive, a galactomannan, and traditional filler materials. The formulation is especially useful where sources of multivalent cations would otherwise cause phase separation and instability of the toothpaste.

CROSS-REFERENCE TO RELATED APPLICATION

This application claims the benefit of priority from Provisional Application Serial No. 60/444,042 filed on Jan. 31, 2003.

### BACKGROUND OF THE INVENTION

1. Field of the Invention

The present invention is directed to a polymeric formulation utilizing a highly carboxylated polymer(s) as a thickening, suspending, or rheology-modifying agent which, in combination with a galactomannan, prevents the premature or undesired precipitation, settling, syneresis, hardening, or solidification of formulations containing sources of multivalent cations.

In particular, the invention is directed to a toothpaste formulation utilizing a cross-linked polymer or copolymer derived from one or more unsaturated carboxylic acids, in combination with a calcium carbonate abrasive, a galactomannan, and traditional filler materials. The toothpaste formulation provides a low cost alternative to conventional counterpart formulations which utilize silica abrasives.

2. Description of the Prior Art

In preparing toothpaste formulations, a thickening material is generally necessary to provide a desirable consistency to the composition. These thickening materials, or “binders” control and/or modify toothpaste rheology, including viscosity, yield value and thixotropy. Of special interest for its ability to impart viscosity at low concentrations to thicken a toothpaste formulation are a number of crosslinked polyacrylic acid polymers and copolymers marketed by Noveon, Inc. under the trade name Carbopol® polymer. Carbopol® polymers possess the ability to build yield value with low thixotropy, thereby providing a clean, non-stringing ribbon of toothpaste.

Despite the well-known benefits of using Carbopol® polymers as a thickening, suspending, or rheology-modifying agent, the wider use of Carbopol® polymer has been limited by its incompatibility with formulations containing multivalent cations, including certain materials utilized as dental abrasives. Suitable dental abrasives in most toothpaste can include, for example, silica gels and precipitates, calcium carbonate, dicalcium orthophosphate, calcium pyrophosphate, tricalcium phosphate, hydrated alumina, and sodium polymetaphosphate, among others. Where cost is an issue, particularly in parts of the world where toothpaste, despite its importance for dental hygiene, remains unaffordable, a preferred dental abrasive is calcium carbonate. However, the use of calcium carbonate with Carbopol® polymer as thickening agent, leads to phase separation and instability of the toothpaste formulation. Furthermore, several other ingredients used in toothpaste, such as stannous fluoride, also cause destabilization of the formulations.

The degradation and storage-instability of carbomer-thickened formulations containing sources of multivalent cations has been observed in other compositions, including calamine and zinc oxide. Historically, formulations thickened using carbomer and containing such ingredients have been stabilized where possible by initial adjustment to a pH greater than 8.5 to 9, thereby suppressing the hydrolysis and solubilization of the multivalent cations. This approach, however, is untenable for most formulations designed for application to living bodies, and for others designed for “delicate substrates”.

### SUMMARY OF THE INVENTION

Specifically, it has now been found that a low cost and traditional abrasive such as calcium carbonate can be used in certain toothpaste formulations thickened using polyacrylic acid polymers and polymers. The multivalent cation formulation consists of a binder of a polymer or copolymer derived from one or more unsaturated carboxylic acids that is cross-linked, cassia gum, and calcium carbonate in conjunction with conventional materials such as sweeteners, humectants, anti-tartar agents, and anti-caries agents.

### DESCRIPTION OF THE INVENTION

The polyacrylic acid polymers or copolymers of the present invention are derived from one or more unsaturated carboxylic acid monomers, (i.e., (di)carboxylic acid) generally having one or two carboxylic acid groups, desirably having one carbon to carbon double bond and containing generally a total of from 3 to about 10 carbon atoms and preferably from 3 to about 5 carbon atoms such as α-β-unsaturated monocarboxylic acids, for example, acrylic acid, methacrylic acid, and crotonic acid, and the like, or dicarboxylic acids such as itaconic acid, fumaric acid, maleic acid, aconitic acid, and the like. Moreover, half ester monomers such as diacids with alkanols containing from 1 to about 4 carbon atoms can also be utilized, such as monommethyl fumarate. Preferred acids include acrylic acid or maleic acid. Additionally, diacids capable of forming cyclic anhydrides, such as maleic, may be polymerized as the anhydride and later reacted with water or alcohols to form the equivalent of maleic acid or monoalkyl maleate copolymer.

Optionally, one or more oxygen-containing unsaturated comonomers having a total of from 3 to about 40 carbon atoms, such as esters of the above unsaturated (di)carboxylic acids, that is, mono or di, especially alkyl esters containing a total of from 1 to about 30 carbon atoms in the alkyl group can also be utilized as comonomers to form the copolymer. Examples of such esters include ethyl acrylate, butyl acrylate, 2-ethylhexyl acrylate, dodecyl acrylate, hexadecyl acrylate, and octadecyl acrylate, and the like, with the C₁₀ to C₃₀ acrylates being preferred.

Another optional class of comonomers are the various anhydrides of the above-noted carboxylic acids such as maleic anhydride, and the like. Moreover, another optional class of suitable comonomers are the various alkyl vinyl ethers wherein the alkyl group contains from 1 to about 20 carbon atoms with examples including ethyl vinyl ether, methyl vinyl ether, and the like. Examples of suitable cross-linked commercially available rheology modifying polymers or copolymers include Carbopol® polymers 956, 974P NF, and 980 NF manufactured by Noveon, Inc.

The amount of the one or more oxygen-containing comonomers when utilized is generally a minor amount, such as from about 0.01% to about 40% by weight, desirably from about 0.5% to about 35% by weight, and preferably from about 1% to about 25% by weight based upon the total weight of all the rheology modifying polymer or copolymer forming monomers and comonomers. Thus, the amount of the one or more unsaturated (di)carboxylic acid monomers, half ester thereof, or combinations thereof, is generally from about 60% to about 99.99% by weight, desirably from about 65% to about 99.5% by weight, and preferably from about 75% to about 99% by weight based upon the total weight of all rheology modifying polymer or copolymer forming monomers or comonomers.

The various polymers or copolymers of the present invention are generally anhydrous. That is, they generally contain 5 parts by weight or less, desirably 3 parts or 2 parts by weight or less, and preferably 1 part or less by weight, and even nil, that is no parts by weight, of water per 100 parts by weight of the one or more rheology modifying polymers or copolymers.

It is an important aspect of the present invention that the polymer or copolymer be cross-linked with one or more polyunsaturated monomers or comonomers. Suitable cross-linking agents are known to the art and literature and generally include the various allyl ethers of sucrose or pentaerythritol, or derivatives thereof, or various polyols. Specific examples include diallylphthalate, diallyl ether, divinyl glycol, divinyl benzene, allyl (meth)acrylate, ethylene glycol di(meth)acrylate, diallyl itaconate, diallyl fumarate, or diallyl maleate. Derivatives of castor oils or polyols such as esterified with an ethylenically unsaturated carboxylic acid and the like can be used. Preferred cross-linking agents include divinyl glycol, allyl ether of sucrose, allyl ether of pentaerythritol, diallylphthalate, and combinations thereof.

The amount of the cross-linking agent is from about 0.001 to about 5 parts by weight, desirably from about 0.02 to about 3.5 parts by weight, and preferably from about 0.03 to about 2 parts by weight per 100 total parts by weight of the one or more rheology modifying monomers or comonomers.

The rheology modifying polymers or copolymers of the present invention are produced by conventional methods known to the art and to the literature such as by dispersion or precipitation polymerization utilizing suitable organic solvents such as various hydrocarbons, esters, halogenated hydrocarbon compounds and the like, with specific examples including aromatic solvents such as benzene, or toluene; various cycloaliphatic solvents such as cyclohexane; various esters such as ethyl acetate and methyl formate, ethyl formate; various chlorinated hydrocarbons such as dichloromethane; and combinations thereof. Preferred solvents generally include benzene, methylene chloride, blends of ethyl acetate and cyclohexane, or ethyl acetate, and the like.

In one embodiment, the rheology modifying polyacrylic acid polymer or copolymers utilized in the present invention will generally be present in an amount from about 0.2% to about 2.0% by weight of the total composition. In another embodiment from about 0.3% to about 1.75%, and in a further embodiment from about 0.5% to about 1.5% by weight of the total composition.

The toothpaste composition of the present invention may optionally contain additional thickening agents, including completely synthetic polymers such as polyoxyethylene, block copolymers of ethylene oxide and propylene oxide (“poloxamer”), and polyvinylpyrollidone; chemically modified natural products such as sodium carboxymethylcellulose, methyl cellulose, hydroxyethyl cellulose, hydroxypropyl cellulose, methyl hydroxypropyl cellulose, and chemically modified starch; as well as natural gums, including xanthan gum, gum karaya, gum Arabic or gum tragacanth, galactomannans, glucomannans, carrageenan, alginates, and the like. When present, these additional thickening agents will generally be present in an amount from about 0.1% to about 2%, and preferably from about 0.2% to about 0.7% by weight of the total composition.

In addition to containing the rheology modifying polymer or copolymer binder, there will be included a galactomannan gum. Galactomannans are vegetable reserve polysaccharides which occur in the endosperm of cells of numerous seeds of Leguminosae. Upon germination of the seeds, they undergo enzymatic degradation and serve as nutrients for the seedling. The collective term “galactomannan” or “polygalactomannan” comprises all polysaccharides which are built up of galactose and/or mannose residues and in addition can also contain minor amounts of other sugar residues. It was unexpectedly found that by adding a galactomannan gum to a formulation containing a divalent cation, and specifically, a toothpaste formulation, phase separation and flocculation of Carbopol® polymer by the divalent cation (e.g., calcium from the calcium carbonate abrasive in toothpaste) was prevented.

The galactomannan gum utilized may be one or more of locust bean gum, guar gum, tara gum and cassia gum. Locust bean gum is obtained from the seeds of the locust bean tree (Ceratonia siliqua L.), which is a native of Mediterranean countries. Guar gum (Guaran) is isolated from the seeds of the guar bean Cyamopsis tetragonolobus L. taub.) which is a native of India and Pakistan. Tara gum is produced in small amounts from the seeds of the tara tree (Cesalipinia spinosa) which grows particularly in Peru. Cassia gum, like locust bean gum, is a pod extract, derived from Cassia tora ubtusifolia, Cassia occidentalis or fistula. The galactomannan gum will generally be present in an amount from about 0.001% to by weight up to about 1% by weight of the total composition, and preferably from about 0.01% by weight up to about 0.25% by weight of the total composition.

An abrasive polishing agent will also be included in the toothpaste formulation of the present invention. Abrasives found in toothpaste function as polishing agents and aid physically brushing during application. Abrasives also participate in the building of toothpaste rheology. Suitable polishing agents include the known calcium-based polishing agents found in toothpaste. These are typically powdered materials having no or very low water solubility and a preferred particle size of about 0.1 to about 40 microns in diameter, more preferably between about 2 to about 20 microns in diameter, with normal particle size distributions. All such agents have polishing activity without being objectionably abrasive. Examples of suitable calcium-based polishing agents include dicalcium phosphate, tricalcium phosphate, calcium carbonate, calcium pyrophosphate, calcium silicate, calcium aluminate, and mixtures thereof. Preferred calcium-based polishing agents are precipitated chalk (calcium carbonate) dicalcium dehydrate, calcium pyrophosphate, tricalcium phosphate, calcium metaphosphate, and mixtures thereof. A preferred abrasive is a precipitate of calcium carbonate having a median particle diameter of 3.00 microns, available from Whittaker, Clark and Daniels, Inc. under the designation 2923-Heavy PCC-USP/FCC.

The surface active agents or surfactants (foaming agents) that may be used in the toothpaste formulation of the present invention are those commonly used to emulsify or otherwise uniformly disperse toothpaste components and facilitate the removal of debris from the oral cavity. Surfactants may include nonionic, anionic, amphoteric, cationic, zwitterionic, or mixtures thereof. Suitable anionic detergents include sodium lauryl sulfate, fatty acid monoglyceride sulfates, fatty alkyl sulfates, higher alkyl aryl sulfonates, higher alkyl sulfoacetates, higher olefin sulfonates, higher aliphatic acrylamides of lower aliphatic aminocarboxylic acids, higher alkyl poly-lower alkoxy (of 3 to 100 alkoxy groups) sulfates, and fatty acid soaps. Examples of these anionic surfactants sodium lauryl sulfate, sodium salt of the monoglyceride monosulfates of hydrogenated coconut oil fatty acids, sodium N-lauroyl sarcoside, and sodium cocate. Suitable types of nonionic detergents include chains of lower alkylene oxides such as ethylene oxide and propylene oxide. However, it is generally preferred that the surfactant be sodium lauryl sulfate or sodium N-lauroyl sarcosinate. The surfactant will generally be present in an amount from about 0.1% to about 5.0% by weight of the total composition, and preferably from about 0.2 to about 2.0% by weight of the total composition.

Additional materials which may be added to the toothpaste formulation include flavorings, sweetening agents, humectants, antitartar agents, anti-caries agents, tooth whitening agents, preservatives and antibacterial compounds. Flavor and sweeteners used in the toothpaste formulation can vary greatly. Examples of flavoring materials can include flavor oils such as spearmint, peppermint, wintergreen, eucalyptus, lemon and lime. Preferred sweeteners include sodium saccharin, glycerin, propylene glycol, sucrose, glucose, dextrose, levulose, mannitol, sorbitol, aspartame, sodium cyclamate and xylitol. When sweetening agents are incorporated into the toothpaste composition, they are generally present in a concentration of about 0.5% to about 60% by weight of the total composition. Suitable humectants which improve consistency and prevent moisture loss, can include sorbitol, xylitol, butylene glycol, polyethylene glycol, propylene glycol, or glycerin, among others. Examples of anti-tartar agents are pyrophosphate salts such as dialkali or tetra-alkali metal pyrophosphate salts including Na₄P₂O₇, K₄P₂O₇, Na₂K₂P₂O₇, Na₂H₂P₂O₇, K₂H₂P₂O₇, long chain polyphosphates such as sodium hexametaphosphate and cyclic phosphates such as sodium trimetaphosphate. Anti-tartar agents, when employed in the composition, are typically present in a concentration of about 0.5% to 5.0% by weight of the total composition. Suitable antibacterials include triclosan. Suitable preservatives include sodium benzoate and methyl and ethyl paraben (Parasept® is a brand name.). Tooth whitening agents that are useful in this invention include calcium peroxide, hydrogen peroxide, urea peroxide, peracetic acid, and sodium percarbonate. Toothpastes designed for sensitive teeth often include potassium nitrate at up to 5% by weight of the total composition.

Either batch or continuous processes may be used to prepare the toothpastes described herein. Such processes are known to those skilled in the art of toothpaste manufacture.

The invention will be better understood by reference to the following examples which serve to illustrate but not to limit the present invention.

### EXAMPLE 1

PREPARATION OF TOOTHPASTE FORMULATION

| Ingredients | Source | % w/w | Actual Wt. (g) |
|---|---|---|---|
| Sorbitol (70%) | Ruger | 43.00 | 172.04 |
| Deionized H₂O | | 30.81 | 123.24 |
| CaCO₃ | Fisher | 14.50 | 58.01 |
| Glycerin | Ruger | 9.60 | 38.40 |
| Sodium lauryl sulfate | Sigma | 0.34 | 1.34 |
| Na monofluorophos-phate | Spectrum | 0.75 | 3.00 |
| Xanthan gum | Kelco | 0.36 | 1.44 |
| Cassia gum | Noveon | 0.04 | 0.16 |
| Carbopol ® 980 NF | Noveon | 0.40 | 1.60 |
| Polymer | | | |
| Na saccharin | Sigma | 0.20 | 0.82 |

Xanthan gum was dispersed into the sorbitol using a Hobart mixer on speed “2” and mixed for 15-20 minutes until smooth. Cassia gum was dispersed in approximately 75% of the DI water and mixed at ˜1000 rpm. The dispersion was heated to 85° C., then cooled to room temperature with constant mixing.

Carbopol® 980NF polymer was slowly dispersed through a screen (20 US Standard mesh) into the cooled cassia solution stirring at about 1000 rpm. The solution of xanthan and the dispersion of cassia and Carbopol® polymer were then placed into a Ross LDM-2 mixer and stirred for 15 minutes at speed level 4 under a vacuum of approximately 25 mm mercury.

Sodium monofluorophosphate and sodium saccharin were dissolved in a portion of the remaining DI water and then added to the glycerin. The glycerin mixture was then added to the Ross mixer and mixed at speed level 4 under vacuum at approximately 25 mm mercury for about 15 minutes. The total amount of calcium carbonate was then added to the mixer and mixed at speed 7 until the mixture appeared smooth, approximately ten minutes.

Sodium lauryl sulfate was added to the remaining DI water and dissolved. The solution was added into the Ross mixer and blended at speed 1-2 under 25 mm mercury vacuum until the mixture stopped foaming and most of the entrapped air had been removed. Upon completion of mixing of the toothpaste, the paste was placed into ½ gallon jars and allowed to set overnight before testing.

| | |
|---|---|
| Appearance | thick, white gel |
| Viscosity | 12,625 cps (20 rpm, #6 spindle) |
| pH (as paste) | 8.56 |
| pH (10% in solution) | 8.9 |
| Stability | 4 weeks at 45° C. |
| Freeze-thaw stability | Passed 10 cycles |

### EXAMPLE 2

TOTHPASTE FORMULATION CONTAINING GUAR GUM

| Ingredients | Soure | % w/w | Actual Weight (g) |
|---|---|---|---|
| Sorbitol (70%) | Ruger | 43.00 | 172.04 |
| Xanthan gum | Kelco | 0.36 | 1.44 |
| DI Water | | 30.81 | 123.24 |
| Guar Gum | Noveon | 0.04 | 0.16 |
| Carbopol ® 980 NF polymer | Noveon | 0.40 | 1.60 |
| Na Monofluorophosphate | Spectrum | 0.75 | 3.00 |
| Na Saccharin | Sigma | 0.20 | 0.82 |
| Glycerin | Ruger | 9.60 | 38.40 |
| CaCO₃ | Fisher | 14.50 | 58.01 |
| Sodium Lauryl Sulfate | Sigma | 0.34 | 1.36 |

Preparation of a toothpaste formulation containing guar gum is as described in Example 1.

| | |
|---|---|
| Appearance | slightly runny, white gel |
| Viscosity | 8,000 cps (20 rpm, #6 spindle) |
| pH (as paste) | 7.26 |
| Stability | 4 weeks at 45° C. |
| Freeze-thaw stability | Passed 10 cycles |

While in accordance with the Patent Statutes the best mode and preferred embodiment have been set forth, the scope of the invention is not limited thereto but rather by the scope of the claims.

What is claimed is:

1. A toothpaste formulation comprising:
a rheology modifying crosslinked polyacrylic acid polymer composition;
a galactomannan gum comprising one or more of guar gum, locust bean gum, cassia gum, and tara gum;
a multivalent cation containing agent;
one or more surface active agents; and
optionally, one or more additional thickening agents.

2. The toothpaste formulation of claim 1 wherein said polyacrylic acid polymer composition is polymerized from at least one unsaturated (di)carboxylic acid monomer having a total of from 3 to about 10 carbon atoms, or at least one half ester monomer of said unsaturated dicarboxylic acid with an alkanol having from 1 to about 4 carbon atoms, or combinations thereof; a crosslinking monomer; and optionally one or more monomers selected from oxygen-containing unsaturated comonomers having from 3 to about 40 carbon atoms and alkyl vinyl ethers where said alkyl group contains from 1 to about 20 carbon atoms.

3. The toothpaste formulation of claim 2 wherein said at least one unsaturated (di)carboxylic acid monomer is selected from acrylic acid, methacrylic acid, crotonic acid, itaconic acid, maleic acid, aconitic acid, monomethyl fumarate, and maleic anhydride.

4. The toothpaste formulation of claim 2 wherein said one or more oxygen-containing unsaturated comomomer is selected from a mono or di alkyl ester of a (di)carboxylic acid where said alkyl group contains from about 1 to about 30 carbon atoms.

5. The toothpaste formulation of claim 4 wherein said mono ester is selected from monomer is selected from ethyl acrylate, butyl acrylate, 2-ethythylhexyl acrylate, dodecyl acrylate, hexadecyl acrylate, and octyldecyl acrylate.

6. The toothpaste formulation of claim 4 wherein said alkyl vinyl ether is selected from methyl vinyl ether, and ethyl vinyl ether.

7. The toothpaste formulation of claim 2 wherein said crosslinking monomer is selected from diallylphthalate, diallyl ether, divinyl glycol, divinyl benzene, allyl(meth)acrylate, ethylene glycol di(meth)acrylate, diallyl itaconate, diallyl fumarate, diallyl maleate, allyl ether of sucrose, allyl ether of pentaerythritol, and combinations thereof.

8. The toothpaste formulation of claim 1 wherein said multivalent cation containing agent is selected from dicalcium phosphate, tricalcium phosphate, calcium carbonate, calcium pyrophosphate, calcium silicate, calcium aluminate, and mixtures thereof.

9. A method for stabilizing a toothpaste composition comprising a crosslinked polyacrylic acid polymer rheology modifier and a multivalent cation containing agent, said method comprising adding a stabilizing amount of a galactomannan gum selected from one or more of guar gum, locust bean gum, cassia gum, and tara gum.

10. The method of claim 9 wherein polyacrylic acid rheology modifier is polymerized from at least one unsaturated (di)carboxylic acid monomer having a total of from 3 to about 10 carbon atoms, or at least one half ester monomer of said unsaturated dicarboxylic acid with an alkanol having from 1 to about 4 carbon atoms, or combinations thereof; a crosslinking monomer; and optionally one or more monomers selected from oxygen-containing unsaturated comonomers having from 3 to about 40 carbon atoms and alkyl vinyl ethers where said alkyl group contains from 1 to about 20 carbon atoms.

11. The toothpaste formualtion of claim 10 wherein said at least one unsaturated (di)carboxylic acid monomer is selected from acrylic acid, methacrylic acid, crotonic acid, itaconic acid, maleic acid, aconitic acid, monomethyl fumarate, and maleic anhydride.

12. The toothpaste formualtion of claim 10 wherein said one or more oxygen-containing unsaturated comomomer is selected from a mono or di alkyl ester of a (di)carboxylic acid where said alkyl group contains from about 1 to about 30 carbon atoms.

13. The toothpaste formulation of claim 12 wherein said mono alkyl ester of said carboxylic acid is selected from monomer is selected from ethyl acrylate, butyl acrylate, 2-ethythylhexyl acrylate, dodecyl acrylate, hexadecyl acrylate, and octyldecyl acrylate.

14. The toothpaste formulation of claim 10 wherein said alkyl vinyl ether is selected from methyl vinyl ether, and ethyl vinyl ether.

15. The toothpaste formulation of claim 10 wherein said crosslinking monomer is selected from diallylphthalate, diallyl ether, divinyl glycol, divinyl benzene, allyl (meth)acrylate, ethylene glycol di(meth)acrylate, diallyl itaconate, diallyl fumarate, diallyl maleate, allyl ether of sucrose, allyl ether of pentaerythritol, and combinations thereof.

16. The toothpaste formulation of claim 1 wherein said multivalent cation containing agent is selected from dicalcium phosphate, tricalcium phosphate, calcium carbonate, calcium pyrophosphate, calcium silicate, calcium aluminate, and mixtures thereof.
